(19) United States
(12) Patent Application Publication
(10) Pub. No.: US 2010/0010606 A1
(43) Pub. Date: Jan. 14, 2010

(54) IMPLANTABLE MEDICAL LEAD INCLUDING A SENSOR

(75) Inventors: Lisa Caffee, Canyon Country, CA (US); Abhi Vase, San Jose, CA (US); Phong D. Doan, Stevenson Ranch, CA (US)

Correspondence Address: PACESETTER, INC., 15900 VALLEY VIEW COURT, SYLMAR, CA, 91392-9221, US

(73) Assignee: PACEQ, Sylmar, CA (US)

(21) Appl. No.: 12/169,504

(22) Filed: Jul. 8, 2008

(51) Int. Cl. A61N 1/362

(52) U.S. Cl. 607/122

(57) ABSTRACT

Disclosed herein is an implantable medical lead. In one embodiment, the lead includes a tubular body, a sensor module, and a distal crimp sleeve. The tubular body includes a distal end and a proximal end. The sensor module includes a sensor and a housing enclosing the sensor. The housing is operably coupled to the tubular body and includes a distal end cap. The distal crimp sleeve is operably coupled to the distal end cap and extends proximally therefrom. The longitudinal axis of the distal crimp sleeve is generally parallel to a longitudinal axis of the housing.

### FIELD OF THE INVENTION

The present invention relates to implantable medical leads and methods of manufacturing and using such leads. More specifically, the present invention relates to implantable medical leads equipped with a sensors and methods of manufacturing and using such leads.

### BACKGROUND OF THE INVENTION

Measurement of physiologic parameters (e.g., pressure, oxygen saturation, etc.) has significant clinical value in the management of heart failure patients. Consequently, it would be highly beneficial to provide implantable medical leads with sensors that allow such physiologic parameters to be accurately monitored.

Unfortunately, there are several complications that adversely impact the accuracy of sensors mounted on an implantable medical lead. For example, if the sensor is located too closely to the lead tip, tissue overgrowth can reduce the accuracy of the sensor. Also, if the sensor ends up being oriented towards or too closely to the heart chamber walls, the sensor accuracy can be reduced. Improper sensor location can lead to inaccurate sensor measurement if the sensor is located in an area of the blood stream that is not well mixed, for example, in the context of oxygen saturation.

There is a need in the art for an implantable medical lead having a sensor that can accurately measure physiologic parameters. There is also a need in the art for a method of using and manufacturing such a lead.

### SUMMARY

Disclosed herein is an implantable medical lead. In one embodiment, the lead includes a tubular body, a sensor module, and a distal crimp sleeve. The tubular body includes a distal end and a proximal end. The sensor module includes a sensor and a housing enclosing the sensor. The housing is operably coupled to the tubular body and includes a distal end cap. The distal crimp sleeve is operably coupled to the distal end cap and extends proximally therefrom. The longitudinal axis of the distal crimp sleeve is generally parallel to a longitudinal axis of the housing.

Disclosed herein is an implantable medical lead. In one embodiment, the lead includes a tubular body and a sensor module. The tubular body includes a distal end, a proximal end and a pre-bend in the tubular body. The center of an arcuate length of the pre-bend is located proximal the distal end by a distance D_{B} of between approximately 18 millimeters and approximately 23 millimeters. The sensor module is operably coupled to the tubular body proximal the pre-bend.

Disclosed herein is an implantable medical lead. In one embodiment, the lead includes a tubular body, a sensor module, sensor cables and means for physically electrically coupling the sensor to the sensor cables. The tubular body includes a distal end and a proximal end. The sensor module is operably coupled to the body and includes a housing and a sensor enclosed by the housing. The sensor cables extend through the tubular body to the sensor module.

Disclosed herein is an implantable medical lead. In one embodiment, the lead includes a tubular body having a distal portion, a proximal end, and a body portion extending between the distal portion and the proximal end. The distal portion includes a distal end, a distal transition sheath proximal the distal end, a sensor module sheath extending proximally from the distal transition sheath, a sensor module enclosed by the sensor module sheath, and a proximal transition sheath extending between the sensor module sheath and a distal end of the body portion.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a diagrammatic view of implantable medical leads extending between treatment sites in a heart and an implantable pulse generator.

FIG. 1B is a diagrammatic overview of an implantable medical lead as employed with the pulse generator in FIG. 1A

FIG. 2A is a side view of a distal portion of the implantable medical lead of FIG. 1B.

FIG. 2B is generally the same view as depicted in FIG. 2A, except of the distal portion of the implantable medical lead is pre-curve.

FIG. 3 is a side view of the sensor module of the lead distal portions depicted in FIGS. 2A and 2B.

FIG. 4 is an isometric side view of the housing assembly of the sensor module of FIG. 3.

FIG. 5 is an isometric side view of a sensor assembly of the sensor module of FIG. 3.

FIG. 6 is a longitudinal cross section of the sensor region of the lead distal portions of FIGS. 2A and 2B.

FIG. 7 is an enlarged view of the proximal end of the longitudinal cross section depicted in FIG. 6.

FIG. 8 is an enlarged view of the distal end of the longitudinal cross section depicted in FIG. 6.

FIG. 9 is a transverse cross section of the sensor region of the lead distal portion as taken along section line 9-9 in FIG. 6.

FIG. 10 is a side view of the sensor module sheath.

FIG. 11 is a transverse cross section of the sensor module sheath, as taken along section line 11-11 of FIG. 10.

FIG. 12 is a side view of the proximal transition sleeve.

FIG. 13 is an isometric side view of the distal transition sleeve.

FIG. 14 is an enlarged side view of the lead proximal portion.

FIG. 15 is a side view of the proximal portion of the lead in the vicinity of the boot, except the boot is shown in phantom line for clarity purposes.

### DETAILED DESCRIPTION

Disclosed herein is an implantable medical lead 5 including a sensor 10 mounted thereon. The lead is configured to optimize the operation of the sensor and to facilitate assembly of the lead and reliable connection between components of the lead. Furthermore, the lead includes transition zones that allow the lead to carry a larger sensor without compromising lead function and implantation.

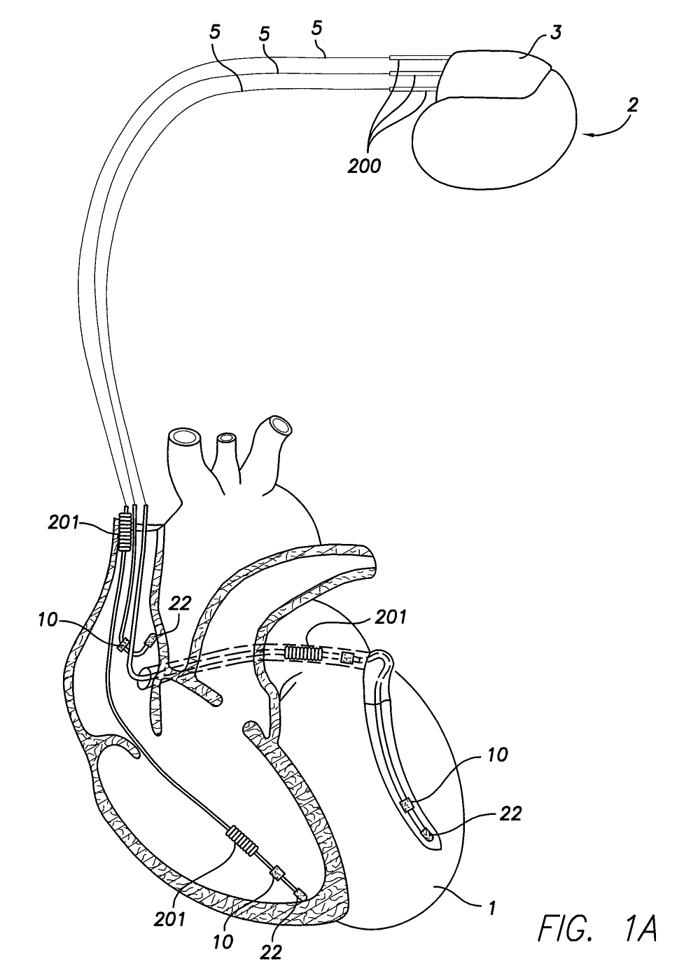

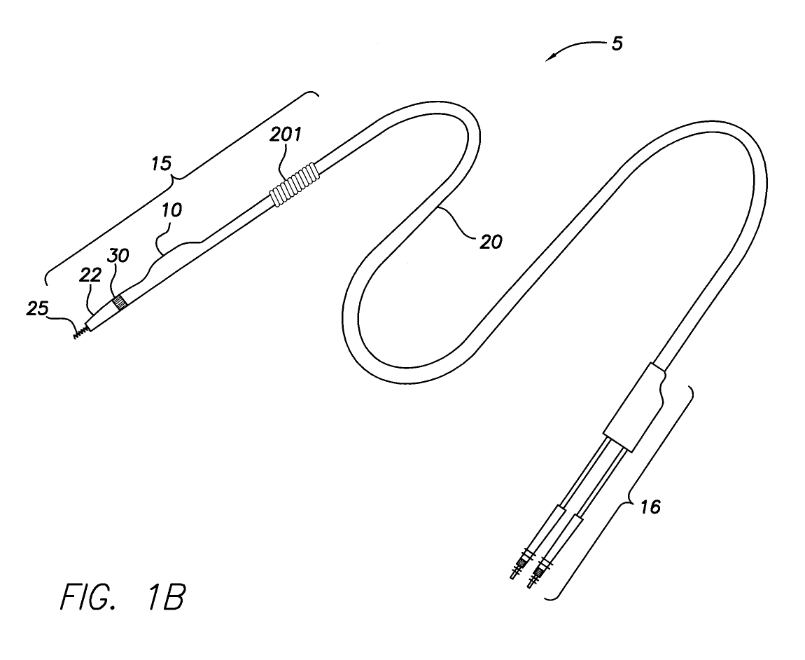

As shown in FIG. 1A, which is a diagrammatic view of implantable medical leads 5 extending between treatment sites in a heart 1 and an implantable pulse generator (e.g., pacemaker, defibrillator or implantable cardioverter defibrillator (“ICD”)) 2, the distal ends 22 of the leads 5 are located within the heart 1, and the proximal ends 200 of the leads 5 are coupled to a header 3 of the pulse generator 2.

As illustrated in FIG. 1B, which is a diagrammatic overview of an implantable medical lead 5 as employed with the pulse generator 2 in FIG. 1A, in one embodiment, a lead 5 will include a tubular body 20 that extends between distal and proximal portions 15, 16 of the lead 5. As can be understood from FIG. 1B and as will be discussed in greater detail later in this Detailed Description, the distal portion 15 of the lead 5 may include a distal end 22, a ring electrode 30 proximal the distal end 22, an anchor 25 extendable from the distal end 22 and a sensor 10. In some embodiments, the distal portion 15 will also include a defibrillation electrode 201 proximal the ring electrode 30. The proximal portion 16 will include a lead connector assembly 205 for mechanically and electrically connecting the lead proximal end to the pulse generator 2. In one embodiment, the lead connector assembly 205 may be an IS-1 connector, DF-1 connector or IS-4 connector.

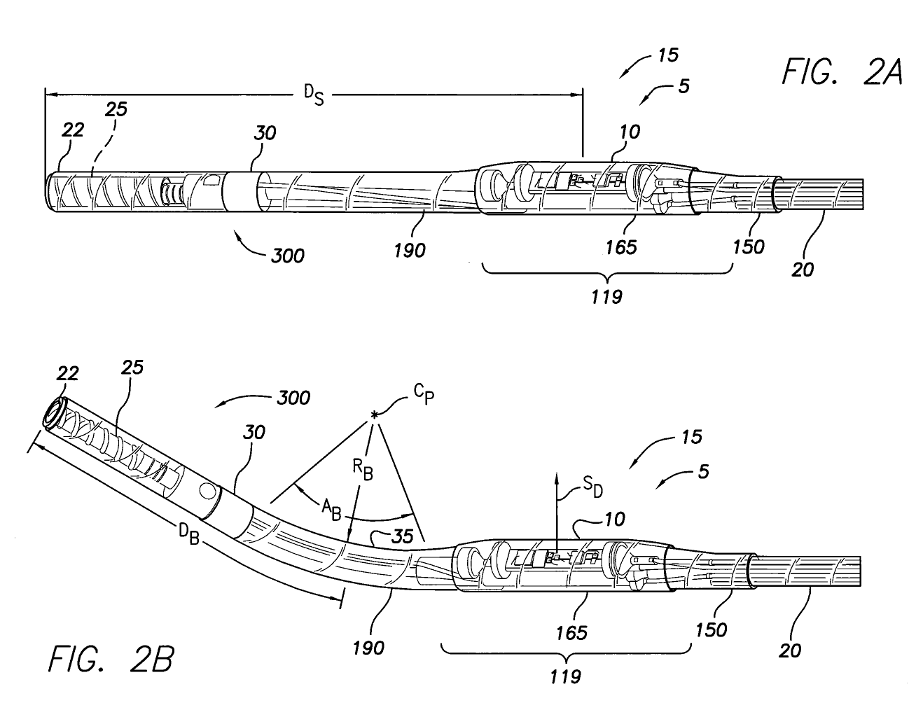

For a more detailed discussion of the configuration of the distal portion 15 of the implantable medical lead 5 of FIG. 1B, reference is made to FIG. 2A, which is a side view of the distal portion 15 of the implantable medical lead 5. As shown in FIG. 2A, in one embodiment, the lead distal portion 15 includes a tubular body 20 with a sensor 10 mounted thereon. The sensor is distally spaced from the distal end 22 of the lead. As indicated via phantom lines in FIG. 2A, in one embodiment, the lead distal portion 15 includes a helical anchor 25 that is extendable from the lead distal end 22 to anchor the distal end to tissue at the implant site. The anchor 25 may be electrically active to act as an electrode for pacing and/or sensing. The lead distal portion 15 may also include a ring electrode 30 proximal of the distal end 22 for pacing and/or sensing. In one embodiment, the longitudinal center of the sensor 10 is proximally offset from the lead distal end 22 by a distance D_{S} of between approximately 25 millimeters and approximately 35 millimeters, or a distance D_{S} of between approximately 30.5 millimeters and approximately 35.5 millimeters.

As shown in FIG. 2A, in one embodiment, the distal portion 15 of the body of the lead 5 is formed during the manufacturing process to be generally straight. Thus, unless acted on by outside forces to assume another shape, the distal portion 15 of the body of the lead 5 will bias into a generally straight shape. Such a lead 5 can be employed, for example, as a RVA or right ventricular outflow tract (“RVOT”) lead.

As depicted in FIG. 2B, which is generally the same view as depicted in FIG. 2A, except of another embodiment, the distal portion 15 of the body of the lead 5 has a bend 35 pre-formed in the lead 5. The bend 35 is formed in the lead 5 during the manufacturing process such that the lead 5 in the region of the bend 35 biases into the bent shape, unless acted on by outside forces to assume another shape. Such a lead 5 can be employed, for example, as a RVA lead.

In one embodiment, the bend 35 is such that its configuration is generally midway between that of a standard straight lead and a J-lead. In one embodiment, the center of the bend 35 is proximally offset from the lead distal end 22 by a distance D_{B} of between approximately 18 millimeters and approximately 23 millimeters. In one embodiment, the center of the bend 35 is proximally offset from the lead distal end 22 by a distance D_{B} of approximately 25 millimeters. In one embodiment, the bend 35 has a bend radius R_{B} of between approximately 10 and approximately 16 and extends over a bend angle A_{B} Of between approximately 50 degrees and approximately 60 degrees. As can be understood from FIG. 2B, the sensing direction S_{D} of the sensor 10 extends from the same side of the lead 5 as the center point C_{P} of the bend 35.

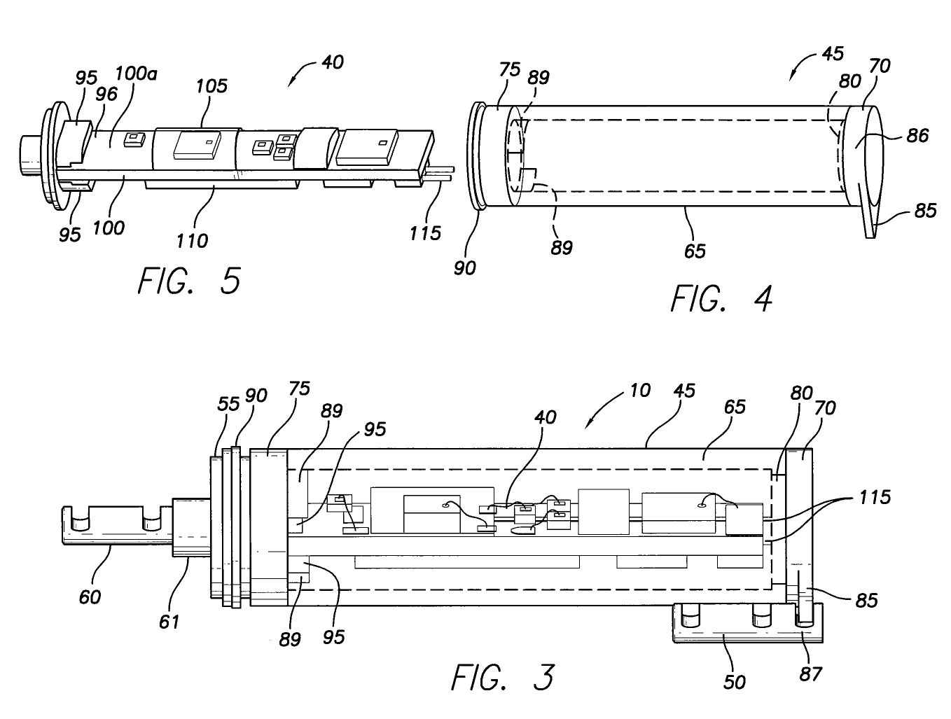

For a discussion regarding the sensor 10 employed on the leads 5 of FIGS. 2A and 2B, reference is made to FIGS. 3-5. FIG. 3 is a side view of the sensor 10 or, more specifically, the sensor module 10. As shown in FIG. 3, in one embodiment, the sensor 10 includes the sensor board 40 (shown via phantom lines), the housing assembly 45, a distal crimp sleeve 50, a proximal end cap 55, and a proximal crimp sleeve 60. The proximal end cap 55 includes a collar 61 generally centered on the proximal face of the cap 55. The distal end of the proximal crimp sleeve 60 is received in the collar 61 such that the crimp sleeve 60 extends proximally from the collar 61. The housing 45 encloses the board 40.

As indicated in FIG. 4, which is an isometric side view of the housing assembly 45 of the sensor module 10 of FIG. 3, the housing includes a cylinder 65, a distal end cap 70, and a proximal end ring 75. The cylinder 65 defines an inner cylindrical volume in which the board 40 is received, as shown in FIG. 3. In one embodiment, the cylinder 65 is a sapphire tube.

As depicted in FIG. 4, the distal end cap 70 includes a raised portion 80 that is circular and extends proximally from the proximal face of the rest of the cap 70. The raised portion 80 is received in the distal end opening of the cylinder 65 so the distal end cap 70 forms the closed distal end of the housing assembly 45. The distal end cap 70 is formed of materials such as Titanium (“Ti”), Niobium (“Nb”), or etc. The distal end cap 70 is brazed (e.g., a gold brazing) or otherwise joined (e.g., via adhesive) to the distal opening of the cylinder 65. A tab 85 radially extends from an outer circumferential surface 86 of the distal end cap 70.

As shown in FIG. 3, the tab 70 is received in a gap 87 of the distal crimp sleeve 50 such that the distal crimp sleeve 50 extends proximally along the outer circumferential surface of the cylinder 65. The longitudinal axes of the cylinder 65 and distal crimp sleeve 50 are generally parallel.

As illustrated in FIGS. 3 and 4, the proximal end ring 75 includes raised portions 89 that include arcuate surfaces and extend distally from the distal face of the rest of the ring 75. The raised portions are received in the proximal end opening of the cylinder 65 so the proximal end ring 75 forms a rim for the proximal end opening of the housing assembly 45. The proximal end of the proximal end ring 75 forms a ring face 90 for brazing, welding, etc. The proximal end ring 75 is brazed (e.g., a gold brazing) or otherwise joined (e.g., via adhesive) to the proximal opening of the cylinder 65. The proximal end cap 55 and end ring 75 are formed of materials such as Ti, Nb or etc. The distal end face of the proximal end cap 55 is welded (e.g., a circumferential weld), brazed or otherwise joined to the ring face 90 of the proximal end ring 75.

As can be understood from FIG. 5, which is an isometric side view of the sensor board 40 of the sensor module 10 of FIG. 3, the proximal end cap 55 includes raised portions 95 distally extending from the distal end face of the end cap 55. A slot is defined between the portions 95 to receive therein the proximal end of the board 40. An electrically conductive epoxy 96 secures the proximal end of the board 40 to the slot.

As depicted in FIG. 3, when the board 40 is received in the cylinder 65 and the proximal end cap 55 is joined to the proximal end ring 75, the raised portions 95 of the cap 55 are received between the raised portions 89 of the ring 75. As can be understood from FIG. 3 and the preceding discussion, the proximal end of the board 40 is placed in electrical communication with the proximal end cap 55 and, as a result, the proximal crimp sleeve 60.

As shown in FIG. 5, in one embodiment, the sensor board 40 includes a printed circuit board (“PCB”) 100, a sensor element 105, a application specific integrated circuit (“ASIC”) 110, various other electrical/electronic components, and connector pins 115. The sensor element 105 is located on an upper side of the PCB 100, the ASIC 110 is located on a bottom side of the PCB 100, and the connector pins 115 extend distally from the distal end of the PCB 100. The pins 115 are formed of materials such as Ti, etc.

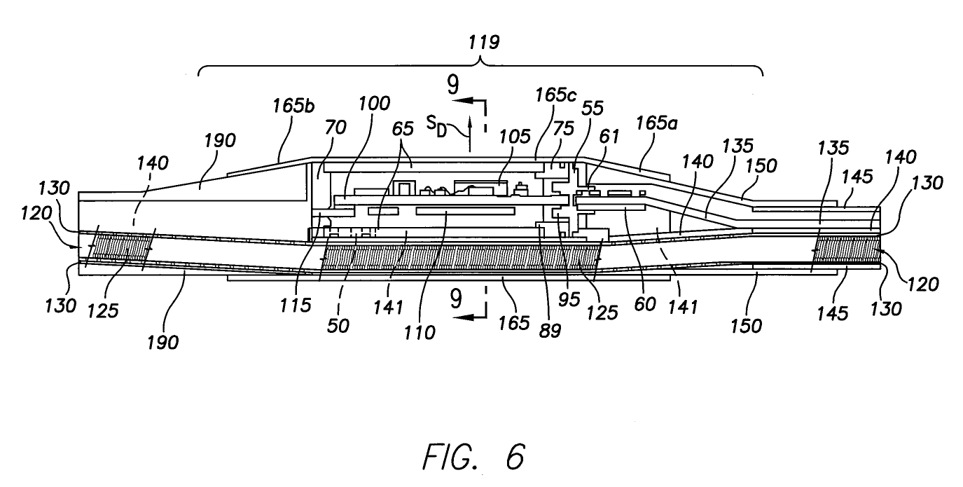

As can be understood from FIG. 3, when the board 40 is received in the volume of the housing 45, the pins 115 extend between the distal end of the board 40 and the proximal face of the distal end cap 70. In one embodiment, as can be understood from FIG. 6, which is discussed below, the proximal face of the distal end cap 70 includes holes for receiving therein the tips of the pins 115. In other embodiments, the tips of the pins 115 simply abut the proximal face of the distal end cap 70. In either case, the tips of the pins 115 are welded, brazed or otherwise connected to the proximal face of the distal end cap 70. As can be understood from FIG. 3 and the preceding discussion, the distal end of the board 40 is placed in electrical communication with the distal end cap 70 and, as a result, the distal crimp sleeve 50.

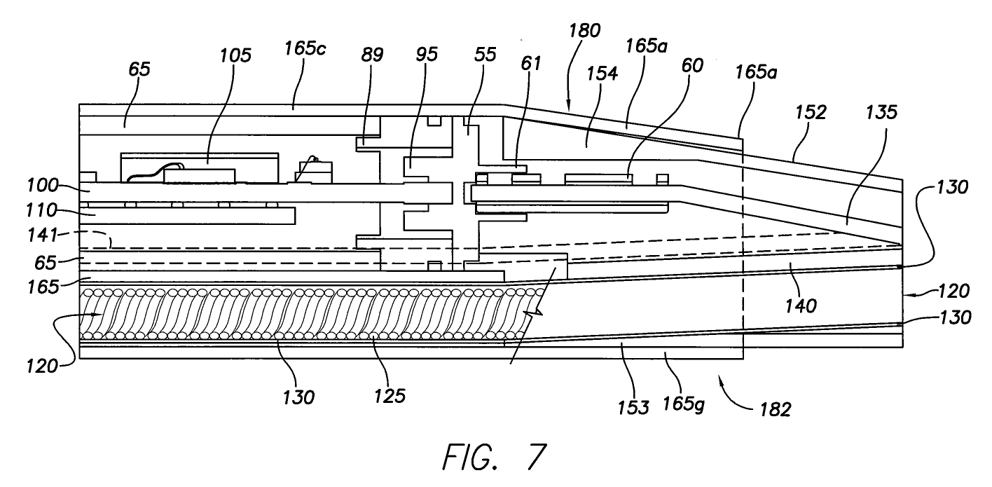

For a discussion regarding the mounting of the sensor module 10 on the body of the lead 5, reference is made to FIGS. 2A-2B and 6-13. FIG. 6 is a longitudinal cross section of the sensor region 119 of the leads 5 of FIGS. 2A and 2B. FIG. 7 is an enlarged view of the proximal end of the longitudinal cross section depicted in FIG. 6.

As can be understood from FIGS. 2A, 2B, 6 and 7, the lead tubular body 20 extends distally from a proximal end of the lead body 20 and includes a stylet lumen 120, a helical coil 125 defining the lumen 120, a polytetrafluoroethylene (“PTFE”) sleeve 130 extending about the coil 125, a first insulated conductor cable 135, a second insulated conductor cable 140, a third insulated cable conductor 141, and an outer layer 145 formed of silicone rubber, polyurethane, or silicone rubber-polyurethane-copolymer (“SPC”). In one embodiment, the lead body 20 forms a quadlumen configuration.

As shown in FIGS. 6 and 7, at the proximal edge of the sensor region 119 of the lead 5, the outer layer 145 of the lead body 20 terminates, but the PTFE sleeve 130, the helical coil 125 and the three conductor cables 135, 140,141 continue to distally extend to and past (in the case of the sleeve 130, coil 125 and second cable 140) the sensor region 119. A proximal end of a proximal transition sleeve 150 extends over the distal termination of the outer layer 145 such that the sleeve 150 overlaps the layer 145. The distal end of the proximal transition sleeve 150 extends to the proximal end cap 55 of the sensor module 10.

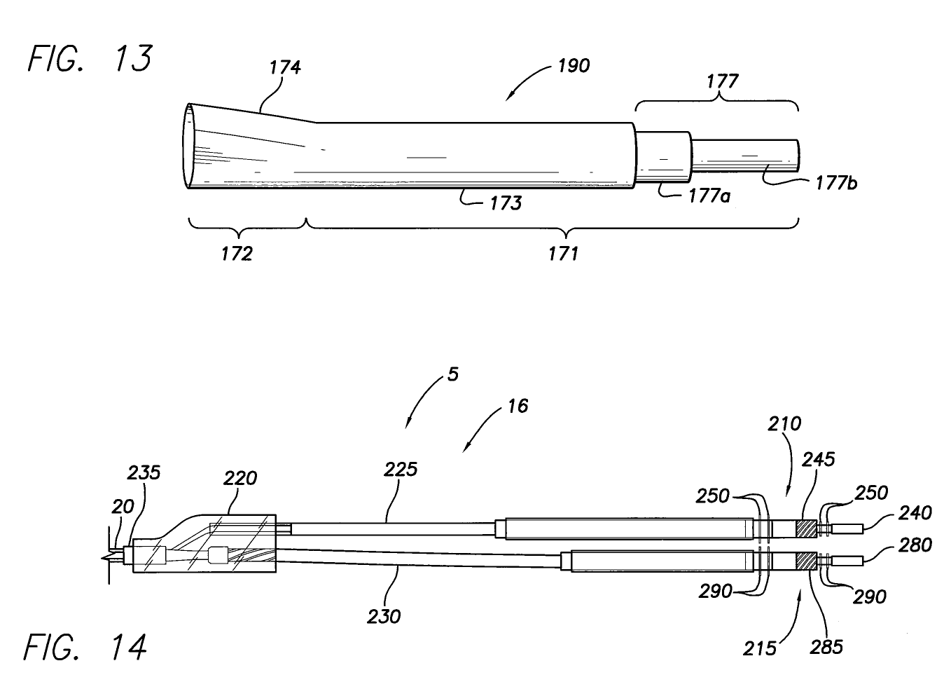

As can be understood from FIG. 12, which is a side view of the proximal transition sleeve 150, the proximal third 151 of the sleeve 150 is generally cylindrical or, in other words, has a generally circular cross section to match the circular cross section of the lead body 20. The distal two thirds 152 of the of the sleeve 150 transitions from the circular cross section of the proximal third 151 to a generally oval-like cross section that gets increasingly taller moving distally along the length of the distal two thirds 152. In other words, moving distally along the length of the distal two thirds 152, the sleeve width (i.e., the dimension of the sleeve 150 normal to the page surface containing FIG. 12) remains generally constant, while the sleeve height (i.e., the dimension of the sleeve 150 transverse to the sleeve width or parallel to the page surface containing FIG. 12) increases until reaching a maximum height at the distal edge of the sleeve 150. As indicated in FIG. 12, the bottom surface 153 of the sleeve 150 extends generally constant (i.e., without diverging) through both regions 151, 152 of the sleeve 150, while the upper surface 154 of the sleeve diverges (i.e., inclines upward) away from the bottom surface 153 in the distal region 152. In one embodiment, the sleeve is molded from silicone rubber, polyurethane or SPC.

As indicated in FIG. 6, the sensor module 10 extends above the coil 125 and its surrounding sleeve 130. The first cable 135 extends into and is crimped in the proximal crimp sleeve 60. The second cable 140 extends along a side of the module 10 as it travels to the ring electrode 30 distal of the sensor module 10. The third cable 141 extends along a side of the module 10 to be received into and crimped in the distal crimp sleeve 70.

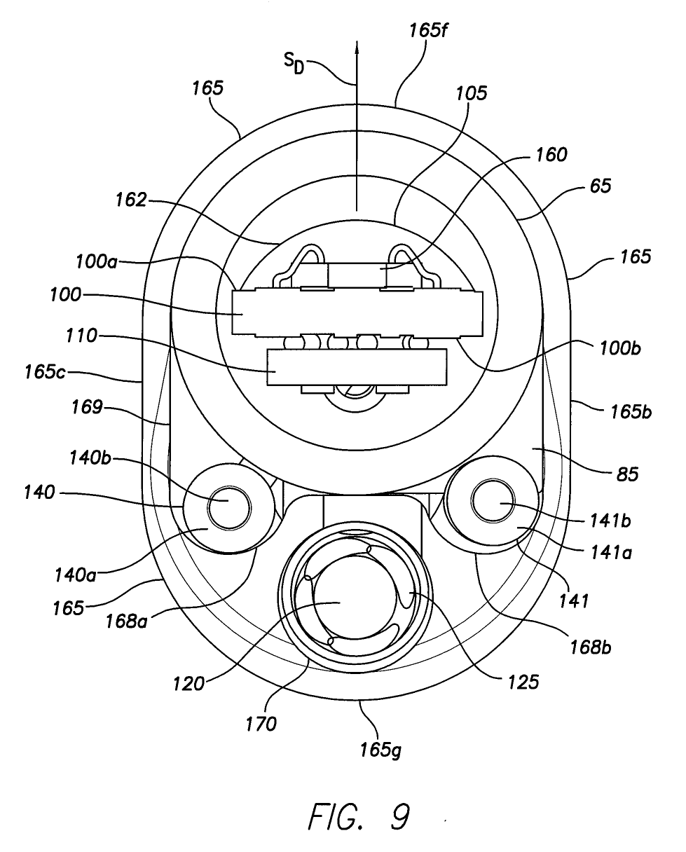

As can be understood from FIG. 9, which is a transverse cross section of the sensor region 119 of the lead 5 as taken along section line 9-9 in FIG. 6, the sensor element 105 is mounted on an upper planar face 100a of the board 100, and the ASIC 110 is mounted on a bottom planar face 100b of the board 100. In one embodiment, the sensor element 105 includes components of an oxygen saturation sensor 40, including light emitting diodes (“LEDs”) 160 and a light barrier 162. In other embodiments, the sensor element 105 will have components of other types of sensors 40, such as a pressure sensor 40, etc. The sensor element 105, ASIC 110, board 100, and various electrical components of the sensor 40 are enclosed within the cylinder 65, which in one embodiment, is a sapphire tube, which has a generally circular cross section.

As indicated in FIG. 9, in one embodiment, the sensing direction S_{D} of the sensor 10 extends generally perpendicularly away from the upper surface 100a of the board 100. In one embodiment, the tab 85 extends in a direction that is approximately 135 degrees from a sensing direction S_{D} of the sensor 10. In one embodiment, the tab 85 extends in a direction that is between approximately 90 degrees and approximately 180 degrees from the sensing direction S_{D} of the sensor 10. In one embodiment, the tab 85 extends in a direction that is between approximately 120 degrees and approximately 150 degrees from the sensing direction S_{D} of the sensor 10.

As indicated in FIG. 9, the second and third cables 140, 140 include electrical insulation jackets 140a, 140b and electrically conductive cores 140b, 141b. In one embodiment, the second cable 140 will extend along the a longitudinal side of the cylinder 65 at approximately 142 degrees counter clockwise, plus or minus approximately 20 degrees, the sensing direction S_{D} of the sensor 10. Similarly, the third cable 141 will extend along the a longitudinal side of the cylinder 65 at approximately 142 degrees clockwise, plus or minus approximately 20 degrees, the sensing direction S_{D} of the sensor 10.

As illustrated in FIG. 9, in one embodiment, the lumen 120, coil 125 and liner 130 are centered laterally between and below the second and third cables 140, 141. The longitudinal center axis of the lumen 120 is centered below the longitudinal center axis of the cylinder 65 at approximately 180 degrees from the sensing direction S_{D} of the sensor 10.

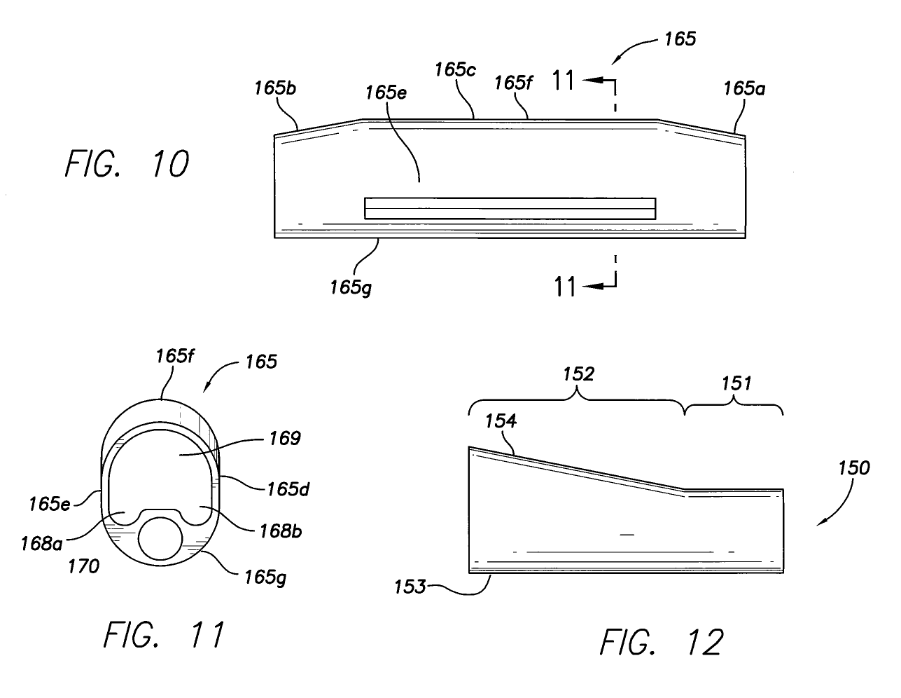

As shown in FIGS. 6 and 9, a sensor module sheath 165 encloses and supports the sensor module 10, the second and third cables 140,141, the lumen 120, coil 125 and sheath 130. As depicted in FIG. 10, which is a side view of the sensor module sheath 165, the sheath 165 includes a tapered proximal end 165a, a tapered distal end 165b, and a center portion 165c having a generally consistent cross sectional area.

As indicated in FIG. 11, which is a transverse cross section of the sensor module sheath 165, as taken along section line 11-11 of FIG. 10, the sheath 165 has a generally oval transverse cross section with generally planar opposed sides 165d, 165e and arcuate upper and lower surfaces 165f, 165g. The sheath 165 includes a primary chamber 168 having two half-circle troughs 168a, 168b and a main area 169. The sheath 165 also includes a lumen 170 centered under the primary chamber 168 between and below the troughs 168a, 168b.

As can be understood from FIGS. 9 and 11, the troughs 168a, 168 receive the second and third cables 140, 141. The main area 169 receives the sensor module 10. The lumen 170 receives the coil 125 and sheath 130.

As depicted in FIG. 13, which is an isometric side view of the distal transition sleeve 190, the distal three quarters 171 of the sleeve 190 is generally cylindrical or, in other words, has a generally circular cross section equal or similar to the circular cross section of the lead body 20. The most distal third 177 of the distal three quarters 171 stepped such that it radially reduces twice to form two reduced diameter cylindrical sections 177a, 177b. In one embodiment, the most proximal reduced diameter section 177a may have a rectangular cross section or at least one radially oriented planar surface. As can be understood from FIGS. 1 and 2, the reduced diameter sections 177a, 177b act as substrates on which the electrode 30 and lead distal end 22 are mounted.

The proximal one quarter 172 of the of the sleeve 190 transitions from the circular cross section of the distal three quarters 171 to a generally oval-like cross section that gets increasingly taller moving proximally along the length of the proximal one quarter 172. In other words, moving proximally along the length of the proximal one quarter 172, the sleeve width (i.e., the dimension of the sleeve 190 normal to the page surface containing FIG. 13) remains generally constant, while the sleeve height (i.e., the dimension of the sleeve 190 transverse to the sleeve width or parallel to the page surface containing FIG. 13) increases until reaching a maximum height at the proximal edge of the sleeve 190.

As indicated in FIG. 13, with the exception of the radially reduced sections 177a, 177b, the bottom surface 173 of the sleeve 190 extends generally constant (i.e., without diverging) through both regions 171, 172 of the sleeve 190. The upper surface 174 of the sleeve diverges (i.e., inclines upward) away from the bottom surface 173 in the proximal region 172. In one embodiment, the sleeve is molded from silicone rubber, polyurethane or SPC.

As can be understood from FIGS. 2A and 2B, in one embodiment, the distal sleeve 190 will a generally straight configuration and, in another embodiment, the distal sleeve 190 will have a curved configuration as discussed above. The straight or curved configuration of the distal sleeve 190 may be a function of the distal sleeve 190 being preformed to bias into a straight or curved configuration. Alternatively, the straight or curved configuration of the distal sleeve 190 may be a function of the coil 125 extending through the distal sleeve region of the lead being preformed to bias into a straight or curved configuration. In one embodiment, the straight or curved configuration of the distal sleeve region may be a function of both the distal sleeve 190 and the coil 125 being preformed to bias into the straight or curved configuration.

As shown in FIGS. 6 and 7, the distal end 152 of the proximal transition sleeve 150 abuts against the proximal face of the proximal end cap 55 of the sensor module 10. The proximal end 165a of the sensor module sheath 165 extends over the distal end 152 of the proximal transition sleeve 150 to overlap the proximal transition sleeve 150. The overlap between the sheaths 165, 150 creates an overlapping bond joint 180, which may be sealed via reflowing methods or an adhesive.

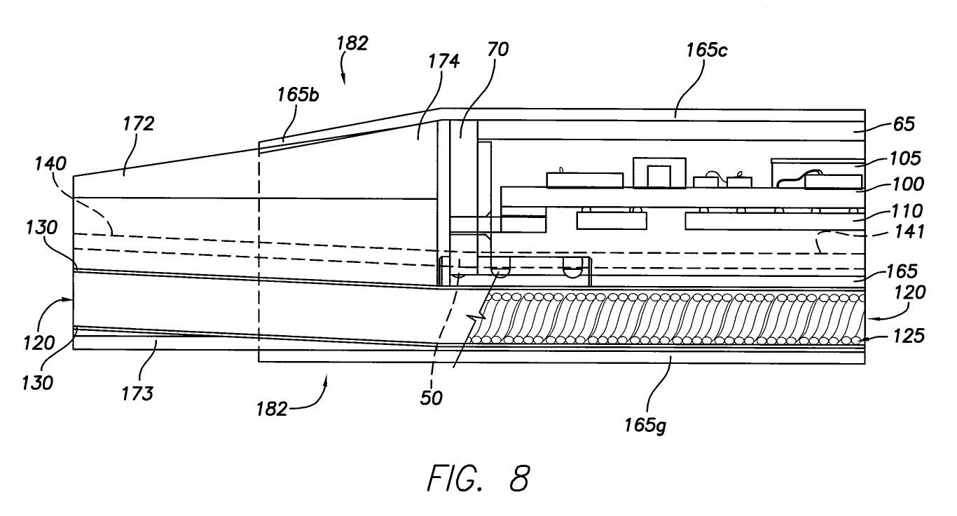

As shown in FIGS. 6 and 8, wherein FIG. 8 an enlarged view of the distal end of the longitudinal cross section depicted in FIG. 6, the proximal end 172 of the distal transition sleeve 190 abuts against the distal face of the distal end cap 70 of the sensor module 10. The distal end 165b of the sensor module sheath 165 extends over the proximal end 172 of the distal transition sleeve 190 to overlap the distal transition sleeve 190. The overlap between the sheaths 165, 190 creates an overlapping bond joint 182, which may be sealed via reflowing methods or an adhesive.

As can be understood from FIGS. 2A, 2B, 6, 8 and 13, the distal transition sleeve 190 extends distally from the sensor region 119. In one embodiment, the distal transition sleeve 190 forms the lead body through which the coil 125, sheath 130, stylet/guidewire lumen 120, and second conductor 140 extend to the lead distal end, and the sleeve 190 serves as the body for supporting the ring electrode 30 and the components forming the lead distal end 22.

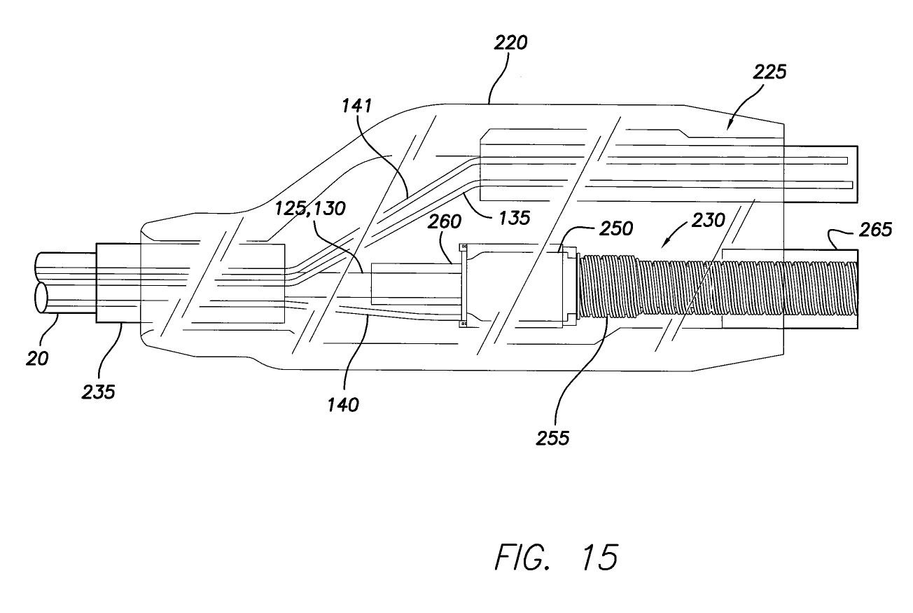

For a discussion regarding the proximal portion 16 of the lead 5, reference is made to FIG. 14, which is an enlarged side view of the lead proximal portion 16. As shown in FIG. 14, the lead proximal portion 16 may be a bifurcated connector assembly 16 that includes a sensor connector assembly 210, an electrode connector assembly 215, and a boot 220. Conductor lines 225, 230 exit the proximal end 225 of the lead body 20 via a silicone sleeve 235 and diverge from each other within the flexible boot 220 as the lines 225, 230 proximally extend to their respective connector assemblies 210, 215. In one embodiment, the lead proximal end may even be considered a bifurcated IS-1 connector assembly 16.

As shown in FIG. 14, the sensor connector assembly 210, which may be configured as an IS-1, IS-4 or similar connector, includes a pin contact 240, a ring contact 245, and seals 250. The contacts 240, 245 make electrical contact with electrical contacts within the header 3 of the pulse generator 2 when the connector 210 is electrically and mechanically coupled to the header 3.

Similarly, the electrode connector assembly 215, which may be configured as an IS-1, IS-4, DF-1 or similar connector, includes a pin contact 280, a ring contact 285, and seals 290. The contacts 260, 265 make electrical contact with electrical contacts within the header 3 of the pulse generator 2 when the connector 215 is electrically and mechanically coupled to the header 3. The pin contact 265 is hollow and leads to the lead body lumen 120, which extends through the line 230 and lead body coil 125 to the lead distal end 22 to receive a guidewire and/or stylet therethrough.

As shown in FIG. 15, which is a side view of the proximal portion 16 of the lead 5 in the vicinity of the boot 220, except the boot 220 is in phantom line for clarity purposes, the sensor cables 135,141 diverge from the coil 125 and ring electrode cable 140 at the proximal edge of the silicone sleeve 235 to be received in a bilumen tubing 225 leading to the sensor connector assembly 210. The sensor connector assembly 210 may be a bipolar IS-1.

As indicated in FIG. 15, the ring electrode cable 140 and coil 125 extend proximally generally parallel to each other from the proximal end of the sleeve 235 to a circumferential cable crimp 250, which is circumferentially welded at its proximal end to an outer coil 255. An inner tubing 260 extends over the sleeve 130 that extends over the coil 125. an outer tubing 265 extends over the outer coil 255. The inner tubing 260, outer coil 255 and outer tubing 265 extend proximally to the electrode connector assembly 215, which may be a Riata®-type connector as manufactured by St. Jude Medical, Inc.

In one embodiment, the overall configuration of the lead distal portion 15 keeps the sensor 10 in a zone where blood is well-mixed and where the sensor 10 is kept off the wall of the heart 1 to reduce the possibility of tissue overgrowth. The configuration can also maintain the orientation of the sensor 10 within the chamber of the heart 1 so the sensor 10 is not facing the wall but is rather facing the lumen of the chamber so as to get the most accurate measurement. The pre-bend also enables effective torque transfer and does not impact the active fixation of the lead 5.

In one embodiment, the sensor module 10, its assembly and its mounting on the lead body 20 results in a sensor equipped lead that is cost effective and reliable.

Although the preceding description of the invention has been made with reference to preferred embodiments of the invention and a best mode presently contemplated for practicing the invention, persons skilled in the art will recognize changes may be made in form and detail without departing from the spirit and scope of the invention. The description of the invention is not to be taken in a limiting sense, but is made merely for the purpose of describing the general principles of the invention. The scope of the invention should be ascertained with reference to the issued claims.

What is claimed is:

1. An implantable medical lead comprising:
a tubular body including a distal end and a proximal end;
a sensor module including a sensor and a housing enclosing the sensor, wherein the housing is operably coupled to the tubular body and includes a distal end cap; and
a distal crimp sleeve operably coupled to the distal end cap and extending proximally therefrom,
wherein a longitudinal axis of the distal crimp sleeve is generally parallel to a longitudinal axis of the housing.

2. The lead of claim 1, wherein a longitudinal side of the distal crimp sleeve extends along a longitudinal side of the housing.

3. The lead of claim 1, wherein the distal end cap includes a radially extending tab.

4. The lead of claim 3, wherein the distal crimp sleeve is operably coupled to the distal end cap by being physically connected to the tab.

5. The lead of claim 3, wherein the tab extends in a direction that is between approximately 90 degrees and approximately 180 degrees from a sensing direction of the sensor.

6. The lead of claim 3, wherein the tab extends in a direction that is between approximately 120 degrees and approximately 150 degrees from a sensing direction of the sensor.

7. The lead of claim 3, wherein the tab extends in a direction that is approximately 135 degrees from a sensing direction of the sensor.

8. The lead as in any claims 5-7, in which the sensor module further comprises a substrate including a planar face on which the sensor is mounted and wherein the sensing direction extends outward from the face generally perpendicularly to the face.

9. The lead of claim 8, wherein the substrate is a printed circuit board.

10. The lead of claim 1, wherein the housing further includes a proximal end cap and further comprising a proximal crimp sleeve proximally extending from the proximal end cap.

11. The lead of claim 10, wherein a longitudinal axis of the proximal crimp sleeve is generally axially aligned with a longitudinal axis of the housing.

12. An implantable medical lead comprising:
a tubular body including a distal end, a proximal end and a pre-bend in the tubular body, wherein a center of an arcuate length of the pre-bend is located proximal the distal end by a distance D_{B} of between approximately 18 millimeters and approximately 23 millimeters; and
a sensor module operably coupled to the tubular body proximal the pre-bend.

13. The lead of claim 12, wherein pre-bend has a bend radius R_{B} Of between approximately 10 and approximately 16.

14. The lead of claim 13, wherein the pre-bend extends over a bend angle A_{B} of between approximately 50 degrees and approximately 60 degrees.

15. The lead of claim 12, wherein a sensing direction S_{D} of the sensor module extends from a same side of the tubular body as a center point C_{P} of the pre-bend.

16. The lead of claim 12, wherein the longitudinal center of the sensing module is proximally offset from the distal end by a distance D_{S} of between approximately 25 millimeters and approximately 35 millimeters.

17. An implantable medical lead comprising:
a tubular body including a distal end and a proximal end;
a sensor module operably coupled to the body and including a housing and a sensor enclosed by the housing;
sensor cables extending through the tubular body to the sensor module; and
means for physically electrically coupling the sensor to the sensor cables.

18. The lead of claim 17, further including sheath means for enclosing the sensor module and operably coupling the sensor module to the body.

19. An implantable medical lead comprising: a tubular body including a distal portion, a proximal end; and a body portion extending between the distal portion and the proximal end, wherein the distal portion includes a distal end, a distal transition sheath proximal the distal end, a sensor module sheath extending proximally from the distal transition sheath, a sensor module enclosed by the sensor module sheath, and a proximal transition sheath extending between the sensor module sheath and a distal end of the body portion.

20. The lead of claim 19, wherein the lead includes a ring electrode mounted on a distal portion of the distal transition sheath.

21. The lead as in any of claims 19 and 20, in which a proximal portion of the distal transition sheath is enclosed by a distal portion of the sensor module sheath.

22. The lead of claim 19, wherein a distal portion of the proximal transition sheath is enclosed by a proximal portion of the sensor module sheath.

23. The lead as in any of claims 19 and 22, in which the distal end of the body portion is received in a proximal portion of the proximal transition sheath.
